(19) United States
(12) Patent Application Publication
(10) Pub. No.: US 2023/0121619 A1
(43) Pub. Date: Apr. 20, 2023

(54) SYSTEM AND METHODS FOR EXAM SUGGESTIONS USING A CLUSTERED DATABASE

(71) Applicant: GE Precision Healthcare LLC, Milwaukee, WI (US)

(72) Inventors: Svein Arne Aase, Trondheim (NO); Beate Ostensen, Horten (NO)

(21) Appl. No.: 17/451,463

(22) Filed: Oct. 19, 2021

(51) Int. Cl. G16H 50/20; G06T 7/00; G16H 30/20

(52) U.S. Cl. CPC G16H 50/20; G06T 7/0012; G16H 30/20; G06T 2207/20092; G06T 2207/30004

(57) ABSTRACT

Methods and systems are provided for providing suggested diagnoses and/or measurements for a patient exam. In one example, a method for a user interface of a medical imaging system includes receiving first user input from a user and determining a first set of measurements on a medical image based on the first user input, sending the first set of measurements to a database of exams, receiving a subset of exams from the database, the subset based on the first set of measurements, determining, after clustering the subset of exams, a second measurement, and suggesting the second measurement to the user before the user performs the second measurement.

### TECHNICAL FIELD

Embodiments of the subject matter disclosed herein relate to ultrasound imaging, and more particularly, to improving the process of identifying clinical findings/diagnosis codes from the measurements of an ultrasound exam.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. Ultrasound images of the internal structures may be saved for later analysis by a clinician to aid in diagnosis and/or displayed on a display device in real time or near real time.

### SUMMARY

In one embodiment, a method for a user interface of a medical imaging system includes receiving first user input from a user and determining a first set of measurements on a medical image based on the first user input, sending the first set of measurements to a database of exams, receiving a subset of exams from the database, the subset based on the first set of measurements, determining, after clustering the subset of exams, a second measurement, and suggesting the second measurement to the user before the user performs the second measurement.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of an ultrasound system;

FIG. 2 is a diagram showing an interface which forms part of the system of FIG. 1;

FIG. 3 is a flow chart illustrating an example method for generating a database;

FIG. 4 is a flow chart illustrating an example method for presenting a user with diagnosis codes and/or findings based on measurements taken during a current exam; and

FIG. 5 is a table illustrating an example database structure that may be generated during a database lookup when determining potential diagnoses and/or measurements to suggest to the user.

FIG. 6 is an example plot of exams clustered in two dimensions.

### DETAILED DESCRIPTION

Some medical imaging systems, such as ultrasound systems, are relatively low cost, non-invasive, and easy to transport, use, and maintain. As such, these medical imaging systems are widely adopted globally. However, in many regions/markets, users of the medical imaging systems may not be experienced with respect to evaluating the images generated by the medical imaging system. For example, while it may be possible to use an ultrasound system to image a patient's heart in a remote or rural location that is far away from a large medical facility, often it may be difficult to find a cardiologist or other experienced and highly trained clinician to evaluate the images and make an accurate diagnosis. Further, even when trained clinicians are available to evaluate the images, some diagnoses may be complex and/or rare, which may result in the clinician having lower confidence in making an accurate diagnosis.

Thus, according to embodiments disclosed herein, possible diagnoses or findings may be automatically suggested based on measurements taken on medical images, such as ultrasound images. The suggested diagnoses/findings may be identified by interrogating a database that includes data from a plurality of prior patient exams. Based on measurements already performed in a current exam, prior patient exams in the database may be identified. In some examples, only prior exams containing at least the same measurements (e.g., the same measurement IDs) as already performed in the current exam are included. Furthermore, if the number of measurements already performed is greater than a threshold, such as greater than six or eight measurements, a dimensionality reduction (e.g. by Principal Component Analysis (PCA)) is performed. The data from the identified prior patient exams is then clustered based on the measurement IDs from the current exam. After the measurement values of the current exam have gone through the same PCA dimensionality reduction (if needed), the current exam can be assigned to one of the clusters of prior patient exams. At this point, the system will find a portion (e.g., the top five most occurring) of the diagnosis codes of the exams in the cluster and suggest these diagnosis codes to the user. Further, a similar process may also be used to suggest one or more additional measurements that may be taken to increase a diagnosis confidence or differentiate between multiple possible diagnoses. Starting with the exams of the database containing at least the same measurements as already in the current exam, each candidate additional measurement (of a list of the most common measurements, e.g., the most common 100 measurements) is considered one-by-one. First, exams also containing the candidate measurement (in addition to the measurements of the current exam) are identified. Then secondly, if indicated, PCA is performed, and then clustering is performed on the identified exams. Once clusters are identified, then a score for the ability of this set of clusters to discern between diagnosis codes is calculated. Once the score is calculated for one or more candidate measurements, the system will suggest the candidate measurement with the highest score. While the original database might originate from a server, the database may be saved in a format that uses a relatively small amount of memory on each device and allows for simple lookups of similar exams, tags, and measurements to provide suggestions for diagnoses and measurements. In this way, once curated and validated, the database may be saved and executed on a variety of devices, such as the medical imaging system itself, which may allow diagnoses and measurements to be suggested to users in a wide variety of clinical settings.

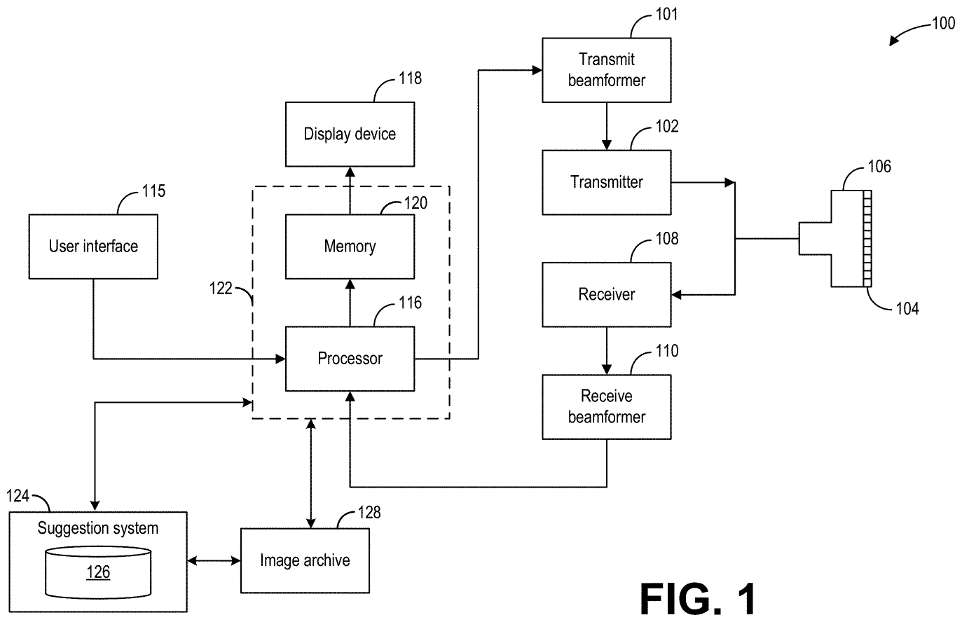

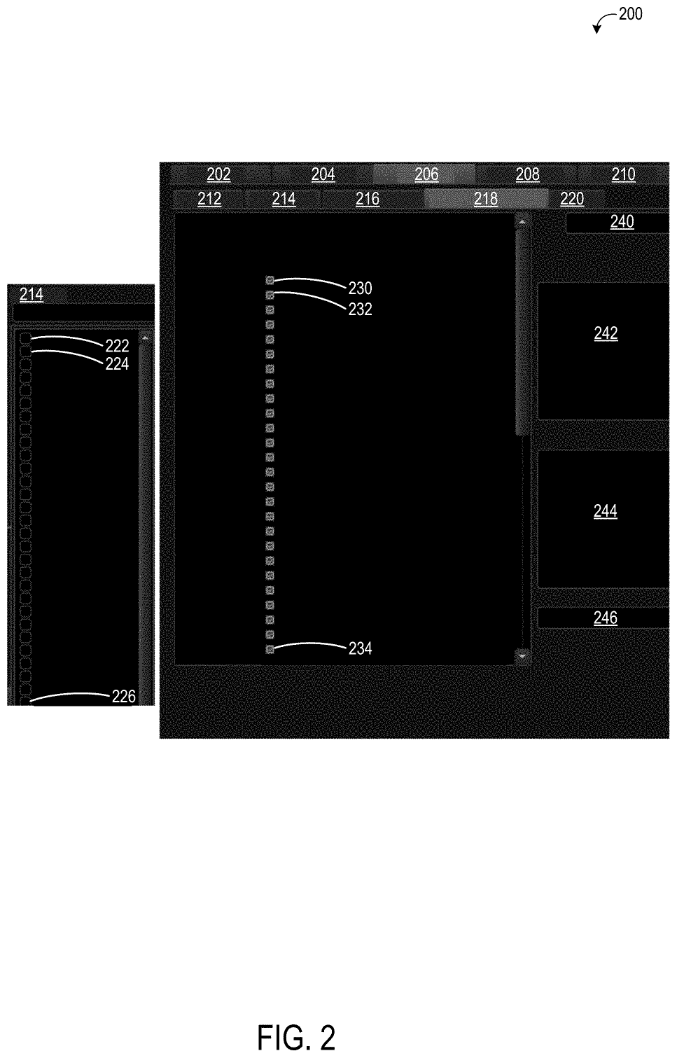

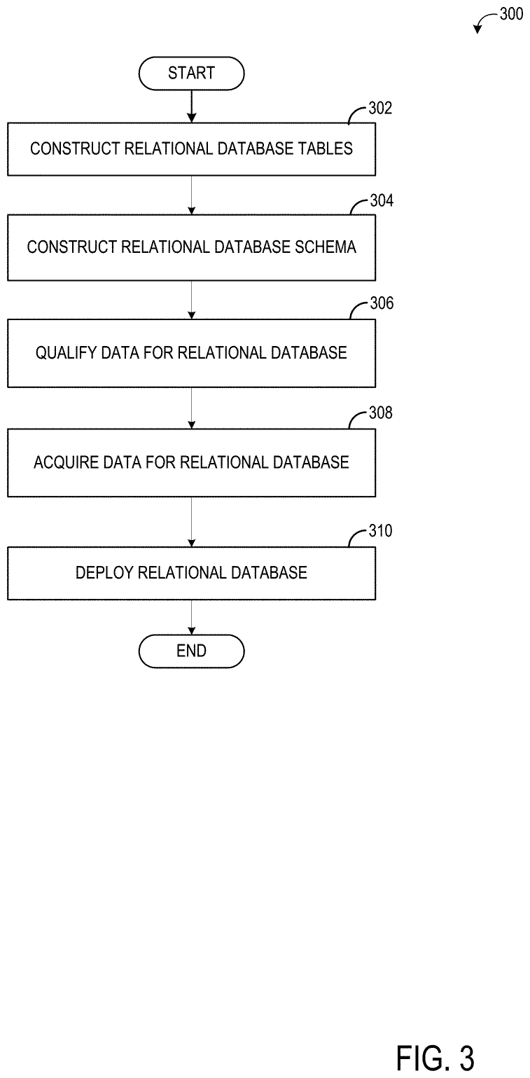

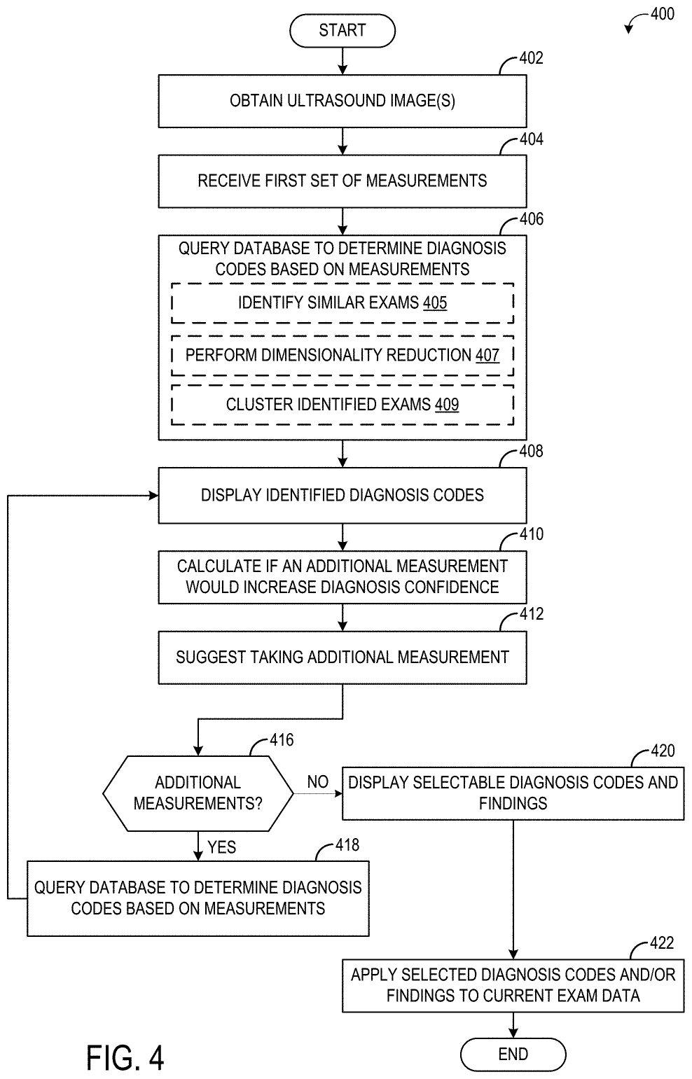

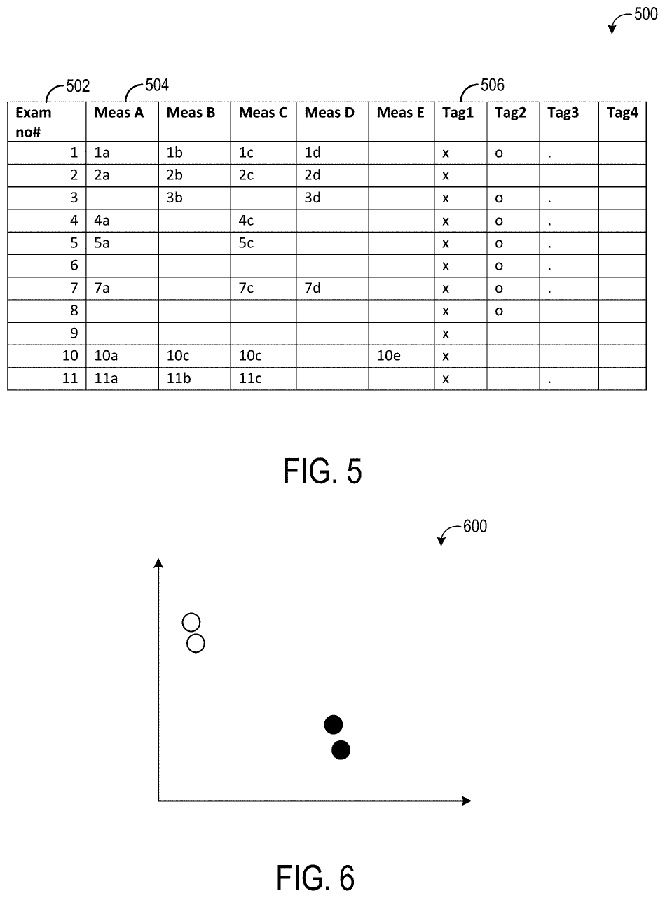

An example ultrasound system including an ultrasound probe, a display device, and an imaging processing system are shown in FIG. 1. Via the ultrasound probe, ultrasound images may be acquired and displayed on the display device. An interface displayed on a display device of FIG. 1 is shown in FIG. 2. A database including medical data may be generated and the data included in the database according to the method of FIG. 3. A user may apply diagnosis codes and/or findings tags to exam data for a current patient based on suggestions made via a clustering analysis of selected exams in the database, according to the method of FIG. 4. An example database structure is shown in FIG. 5, illustrating potential calculations performed during a database lookup during user operation of the system, based on clustering of exams as illustrated in FIG. 6.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic wave. In this way, transducer elements 104 may convert electronic transmit signals into acoustic transmit beams.

After the elements 104 of the probe 106 emit pulsed ultrasonic signals into a body (of a patient), the pulsed ultrasonic signals reflect from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data.

The echo signals produced by transmit operation reflect from structures located at successive ranges along the transmitted ultrasonic beam. The echo signals are sensed separately by each transducer element and a sample of the echo signal magnitude at a particular point in time represents the amount of reflection occurring at a specific range. Due to the differences in the propagation paths between a reflecting point P and each element, however, these echo signals are not detected simultaneously. Receiver 108 amplifies the separate echo signals, imparts a calculated receive time delay to each, and sums them to provide a single echo signal which approximately indicates the total ultrasonic energy reflected from point P located at range R along the ultrasonic beam oriented at angle θ.

The time delay of each receive channel continuously changes during reception of the echo to provide dynamic focusing of the received beam at the range R from which the echo signal is assumed to emanate based on an assumed sound speed for the medium.

Under direction of processor 116, the receiver 108 provides time delays during the scan such that steering of receiver 108 tracks the direction θ of the beam steered by the transmitter and samples the echo signals at a succession of ranges R so as to provide the proper time delays and phase shifts to dynamically focus at points P along the beam. Thus, each emission of an ultrasonic pulse waveform results in acquisition of a series of data points which represent the amount of reflected sound from a corresponding series of points P located along the ultrasonic beam.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the real RF data and generates complex data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

The processor 116 and memory 120 may be included in a computing device 122. Computing device 122 may be a local device configured to be positioned in the same room/area as the probe 106 and may be coupled to the probe 106 via a wired or wireless connection. The computing device 122 may include a communication subsystem that may allow computing device 122 to communicate with additional external computing devices. As shown, computing device 122 is communicatively coupled to a suggestion system 124 and an image archive 128. Suggestion system 124 may be a computing device having resources (e.g., memory, processors) allocated to building and utilizing a database of clustered measurements (referred to herein as database 126). As will be explained in more detail below, via the database 126, the suggestion system 124 may provide suggestions for diagnosis codes, findings, and/or additional measurements to be taken for a patient exam that includes medical images, such as ultrasound images generated by ultrasound system 100. The database 126 may be populated with data received from image archive 128, for example. Image archive 128 may be a picture archiving and communication system (PACS), a vendor neutral archive (VNA), or another suitable storage system configured to store patient exams. While not shown in FIG. 1, information stored on image archive 128 may be accessible through a separate computing device, referred to as a workstation, that may have a display device, user input devices, etc.

While FIG. 1 shows the ultrasound computing device (e.g., computing device 122), the suggestion system 124, and image archive 128 as separate devices, it is to be understood that in some examples, one or more of the devices may be combined in a single device. For example, the suggestion system 124 may reside on the image archive 128. Further, in some examples, the database 126 may be included as part of a separate device or the database 126 may be included as part of the image archive 128. In still further examples, aspects of suggestion system 124 may be included on computing device 122. For example, after database 126 has been built and prior patient exams with associated measurements, diagnosis codes/findings have been curated, the mapping of the exams in multi-dimensional space and/or the structured database format as shown in FIG. 5 may be stored locally on the computing device 122 and the computing device 122 may be configured to provide suggestions for diagnosis codes, findings, and measurements based on the mapping.

Turning now to FIG. 2, it shows an embodiment of an interface 200 that may form part of the system of FIG. 1. In one example, the interface 200 may be displayed on a display device such as display device 118 of FIG. 1, or on a separate display device communicatively coupled to a storage device configured to save medical images, such as a PACS workstation. Interface 200 may display a plurality of diagnosis codes, findings, and/or tags to a user, allowing the user (e.g., a clinician) to select any amount of diagnosis codes, findings, and/or tags to be included as part of a current patient exam. As used herein, a patient exam may include one or more medical images of a patient, such as one or more ultrasound images, and associated diagnosis codes, findings, tags, and/or measurements that are selected, performed, or otherwise applied by a clinician. To complete the patient exam, the clinician may analyze the one or more medical images, perform measurements of anatomical features present within the one or more medical images, and use the interface 200 to associate diagnosis codes, findings, and/or tags with the one or more medical images, which may all be saved as part of the patient exam. A patient exam may also be referred to herein as a patient report.

Menu buttons, such as first menu button 202, second menu button 204, third menu button 206, fourth menu button 208, and fifth menu button 210, may represent selectable menus the user may choose when interacting with the system, labeled accordingly. A selected menu may be visually indicated by a color change, such as third menu button 206. In one example, third menu button 206 may be a menu for reports, where the user may view additional menus/submenus in order to select diagnosis codes, findings, etc., to be included in the report.

Submenu buttons, such as first submenu button 212, second submenu button 214, third submenu button 216, fourth submenu button 218, and fifth submenu button 220, may represent selectable submenus the user may choose when interacting with a selected menu of the system, labeled accordingly. A selected submenu may be visually indicated by a color change, such as fourth submenu button 218.

In one example, second submenu button 214 may be a submenu for diagnosis codes, where a list of available/selectable diagnosis codes may be displayed when the second submenu button 214 is selected. All diagnosis codes, including a first diagnosis code 222, a second diagnosis code 224, and an Nth diagnosis code 226 may be displayed, where N may be a number of total diagnosis codes in the diagnosis codes submenu. If the user selects one of the diagnosis codes, that diagnosis code may be saved as part of the patient exam/report. The diagnosis codes may include diseases, disorders, symptoms, or other clinically-relevant observations, and in some examples may be defined by national or international regulatory/governing bodies, such as ICD codes. In some examples, the user may specify the type of exam being conducted (e.g., an echocardiogram) via the interface 200, and a subset of possible diagnosis codes related to the exam type may be displayed.

In one example, fourth submenu button 218 may be a submenu for findings, where a list of findings may be displayed upon the fourth submenu button 218 being selected, allowing the user to look through finding tags that may be selected and applied to the report. All finding tags, including a first finding tag 230, a second finding tag 232, and an Mth finding tag 234 may be displayed, where M may be a number of total finding tags in the findings submenu. Findings may be similar to diagnosis codes and thus indicate diseases, disorders, symptoms, etc. Findings may be user-specified and/or hospital-specified and may include findings drawn from diagnosis codes as well as additional patient information, such as patient history. Similar to the diagnosis codes, the list of findings that is displayed may be based on the type of exam being performed.

In some examples, a user may be able to specify a new finding or include additional information about an existing finding by entering information into additional boxes, including a label box 240, a findings text box 242, a conclusion text box 244, and a billing code box 246. The user may enter input to label box 240 to define a display label (e.g., name) for a finding, where label 240 may display anywhere a findings tag may be displayed as a representation of the findings tag. Via the findings text box 242, the user may enter a detailed description of a findings tag, such that any information relating to, associated with, or further detailing a findings tag may be included. Via the conclusion text box 244, the user may enter guided diagnosis information regarding possible diagnoses or conclusions to make about the patient based on the medical images and patient history with the associated findings tag. In one example, information entered via the conclusion text 244 of a findings tag may include a plurality of diagnoses for the user to consider based on the information associated with the findings tag. Billing code 246 may include related billing codes to apply to the current patient exam based on the associated findings tags.

If the user chooses to add a user defined findings tag, the user may fill out label 240, findings text 242, conclusion text 244, and billing code 246 to apply the user defined findings tag to the system. Further, while not shown in FIG. 2, via interface 200, medical images may be displayed and measurements may be performed and saved via interface 200. For example, an image of a heart may be displayed and a user may measure the thickness of the interventricular septum (IVS) of the heart via one or more user inputs (e.g., the user may place a first measurement point on a first side of the IVS and place a second measurement point on a second side of the IVS and the thickness may be measured as the distance from the first point to the second point). These measurements may be saved as part of the patient exam/report.

Thus, interface 200 may be displayed during the analysis stage of a patient exam where medical images may be reviewed by a clinician such as a cardiologist to confirm or rule out one or more patient conditions, diseases, disorders, etc. In order to select a diagnosis code or finding, the clinician may perform one or more measurements of anatomical features present in the medical images and choose one or more diagnosis codes and/or findings based on the measurements. For example, the patient exam may be an echocardiogram (also referred to herein as an echo) and the medical images may include a plurality of ultrasound images of the patient's heart, in various standard views, including Doppler imaging. The clinician may review the medical images and take uniquely identifiable measurements, such as distance measurements, area measurements, velocity measurements, etc., of various features of the heart, such as the left ventricle, right ventricle, interventricular septum, blood flow, etc. In certain exams such as echoes, the number of different measurements that may be taken is relatively large (20 or greater measurements taken from a larger possible number of measurements, such as 100 possible measurements) and the number of different diagnosis codes and findings that may be available for selection may also be relatively large, such as 5 or more diagnosis codes and/or findings. Each clinician may choose to take different measurements and may draw different conclusions from the measurements. Further, some clinicians may rely on visual assessment rather than taking measurements.

Accordingly, the amount of time for performing a patient exam may be lengthy, and the lack of standardized protocols for performing the patient exam may result in inconsistent patient diagnoses, particularly by inexperienced users. The sheer volume of possible measurements that may be performed in echoes or other complex exams may present a challenge for inexperienced users, who may not be aware of which measurements may best indicate a given diagnosis, or which diagnosis to make given the large number of available measurements.

Thus, as described herein, during a patient exam where measurements of anatomical features present in medical images are taken in order to select one or more diagnosis codes and/or findings, suggestions may be provided for subsequent measurements and/or diagnosis codes/findings based on one or more prior measurements. The suggestions may be generated based on exams in a database of prior exams being clustered (potentially after dimensionality reduction) for the specific measurements (e.g., based on the measurement IDs) of the current exam, such as database 126 of FIG. 1. The database may include measurements from a plurality of prior patient exams that are clustered via a non-supervised clustering algorithm, such that each exam is associated with one or more clusters of similar exams, based on the measurements performed in the exams and the values of those measurements. The process for suggesting diagnosis codes/findings or additional measurements is detailed below.

FIG. 3 shows a flow chart illustrating an example method 300 for constructing a relational database of medical information from which exams may be identified and a clustering algorithm executed on the medical information (e.g., exams) included in the database. Method 300 is described with regard to the systems and components of FIGS. 1-2, though it should be appreciated that the method 300 may be implemented with other systems and components without departing from the scope of the present disclosure. Method 300 may be carried out according to instructions stored in non-transitory memory of a computing device, such as memory 120 and processor 116. In other examples, method 300 may be carried out by a computing device having non-transitory memory and one or more processors and in communication with the ultrasound system of FIG. 1 and/or an image archive, such as suggestion system 124 of FIG. 1.

At 302, method 300 includes constructing relational database tables that will include data from a plurality of prior patient exams. The relational database tables may be an internal database (e.g., internal to a specific hospital or other medical facility) constructed according to guidelines a hospital or medical facility may adhere to, and thus at least in some examples the information from the plurality of prior patient exams included in the relational database may be extracted/obtained from only that hospital or medical facility. In other examples, the plurality of prior patient exams included in the relational database may be extracted/obtained from more than one hospital or medical facility. Database tables that may be constructed include an examination table, a measurement table, a diagnosis code table, and a findings table, though it will be appreciated that any amount of database tables may be constructed to include relevant medical data as it relates to embodiments of this disclosure. The examination table may include identifying information for each of the plurality of prior patient exams (e.g., exam type, such as echocardiogram, fetal ultrasound, etc.). In one example, the measurements database table may include all measurements taken in each of the plurality of prior patient exams, including the values of each measurement, with possible associated information for each measurement including a doctor taking the measurement, a date the measurement is taken, a unit of measurement, and the like. The diagnosis code table and findings table may each include the diagnosis codes and findings tags, respectively, from each of the plurality of prior patient exams. In some examples, the diagnosis code table and the findings table may be combined into one table.

At 304, method 300 includes constructing relational database schema. In one example, the measurement database table may have a many to many relationship with the diagnosis codes database table, the measurement database table may have a many to many relationship with the findings database table, and the diagnosis codes database table may have a many to many relationship with the findings database table.

At 306, method 300 includes qualifying data for the relational database.

Data (e.g., from one or more prior patient exams) may be acquired from external sources (e.g., other hospitals) to aggregate with the internal relational database. Qualifying data from external sources may include checking a consistency of user defined tags, such as user defined findings tags. For example, different hospitals may follow different standards/protocols for naming findings and thus some findings may have different names depending on the hospital from which the exam was obtained. In one example, if the data is acquired from one or more hospitals external to the internal hospital, user defined tags may be allowed to be included and aggregated with the relational database if the user defined tags are consistent with the nomenclature used in the internal hospital (e.g., originally included in the relational database). To determine if the user defined tags are consistent with the internal hospital nomenclature, each user defined tag from the external sources may be compared to the tags from the internal source (e.g., the internal image archive), and if a given tag from the external source matches a tag from the internal source, that tag may be determined to be consistent with the internal nomenclature. A similar approach may be taken to determine if user defined tags from the internal source are consistent with each other. For example, user defined tags that are recently-used tags (e.g., used within the past three months) may be identified as consistent and/or user defined tags that are frequently used tags (e.g., used more than 5 times) may be identified as consistent. In another example, if the data is acquired from more than one hospital, user defined tags may not be included and aggregated with the relational database due to a likelihood that user defined tags may not be consistent among the multiple hospitals. Rather, only tags that are known to be consistent (e.g., machine-based tags, ICD codes) may be used. Qualification metrics may further include quantifying a number of patient exams that may be included, quantifying a number of patient exams with diagnosis codes tags, findings tags, and the like that may be included, quantifying a number of measurements taken per patient exam that may be included, and quantifying a number of patient exams that may be signed off (e.g., a user tagged a patient exam as complete) that may be included. In one example, patient exam data that may qualify to be included in the relational database may include patient exams with 4 or fewer associated diagnosis codes and/or 8 or fewer associated findings, patient exams with between 10 and 200 associated measurements, and patient exams tagged as complete by a user, meaning any patient exam data not meeting these qualifications may not be included in the relational database.

At 308, method 300 includes acquiring the data for the relational database. The data may include a subset or all of the non-image data from the plurality of prior patient exams. Contents of any external database that may be acquired may be converted by a plurality of scripts to a serializable and transferrable representation. In this way, the database tables described above may be populated with the data from the plurality of prior patient exams.

At 310, method 300 includes deploying the relational database for access on and/or from one or more computing devices. Once the relational database has been built, the database may be queried in order to identify possible diagnosis codes and/or measurements, based on measurements already performed in a current exam. Additional details on how the database is deployed to suggest diagnosis codes and/or measurements is provided below with respect to FIGS. 4 and 5.

In some examples, prior to executing the clustering algorithm, the measurement data in the relational database may be pre-processed to normalize the measurement values according to patient body surface area (BSA), age, and/or gender. Alternatively, separate clustering may be performed for each of a plurality of gender and/or age groups (e.g., pediatric versus adult). Further, separate clustering may be performed for each type of exam. For example, all echoes may be clustered as one group, while all fetal ultrasounds may be clustered as another group.

FIG. 4 shows a flow chart illustrating an example method 400 for suggesting diagnosis codes and/or measurements to a user based on measurements in a patient exam for a current patient based on measurements taken and related patient exam data from a database, such as the database 126 of FIG. 1 and/or the relational database described above with respect to FIG. 3. Method 400 may be carried out according to instructions stored in non-transitory memory of a computing device, such as memory 120 storing instructions executable by processor 116. In other examples, method 400 may be carried out by a computing device having non-transitory memory and one or more processors and in communication with the ultrasound system of FIG. 1 and/or an image archive and with the relational database, such as a PACS workstation or clinical device.

At 402, method 400 includes obtaining ultrasound images from a current patient exam. Ultrasound images may be acquired using an ultrasound system, such as the ultrasound imaging system of FIG. 1. In some examples, the ultrasound images may be acquired and displayed while method 400 is executed, such that measurements are taken and diagnosis codes and/or findings are selected for the exam at substantially the same time the images are acquired. In other examples, the ultrasound images may be obtained from an image archive (e.g., a PACS) after the imaging session with the patient is complete. The ultrasound images may be displayed on an exam interface, such as the interface 200 described above with respect to FIG. 2.

At 404, method 400 includes receiving a first set of measurements. When the ultrasound image is displayed on the display device, the user may make measurements of distance, velocity, area, volume, frequency, and/or the like of one or more anatomical features in the displayed ultrasound image(s), via user input received at the computing device. When a sufficient number of measurements have been taken (e.g., five or more), the measurements may be used to suggest diagnosis codes and/or measurements, as explained below. In some examples, the user may look at the ultrasound image on the display device and not make a measurement, opting to make a diagnosis or add associated information on only visual analysis. In such examples, the user may select from selectable diagnosis codes that are displayed via the exam interface. The selectable diagnosis codes (and/or findings) may be displayed at any time during the exam in response to a user request, or the selectable diagnosis codes and/or findings may be displayed along with the ultrasound image(s) on the exam interface for the entire duration of the exam.

At 406, method 400 queries the database to determine diagnosis codes to suggest based on the first set of measurements. The diagnosis codes may be associated with related or equivalent measurements in prior exams and identified based on the measurements of the current exam. An example output from the query is illustrated and described below with respect to FIG. 5. Related or equivalent measurements may include any/all measurements from previous exams with matching measurement identifications (e.g., the same measurement but not necessarily the same measured value). In one example, a user may be a specialist specializing in one specific area of the body, so related measurements may include any measurements taken by the user from previous patient exams. In another example, related measurements may be included if the previous patient exam occurred within a maximum amount of time before the current patient exam.

For example, the measurements of the current exam (so far) are used to extract only the exams of the database containing the same measurement identifications (not necessarily same measured values), as indicated at 405. For example, if the current measurements include a first measurement of intra ventricular septum diameter in 2D in diastole (IVSd) and a second measurement of left ventricular ejection fraction (EF), exams that also include measurements of IVSd and EF may be identified and extracted, regardless of the values of the measurements. If the number of measurements in the current exam is relatively large (e.g., greater than six measurements), dimensionality reduction by principal component analysis (PCA) is performed, as indicated at 407. The identified exams are clustered, as indicated at 409. The clustering may be non-supervised clustering (e.g. by k-nearest neighbors/k-means) that is performed on the top PCA dimensions (e.g., the top 5 PCA dimensions). At this point, the measurements of the current exam can be converted into corresponding PCA values which are used to identify which cluster(s) of the clustered exams from the database to which the current exam most closely corresponds. Then, based on the top most common diagnosis codes, findings, and/or tags occurring in the identified cluster, these diagnosis codes, findings, and/or tags can be presented to the user of the current exam.

It is to be appreciated that to identify a cluster/prior patient exam with high confidence, more than one measurement may be required. Thus, the process described above (e.g., identifying similar prior patient exams based on the first measurements) may be iteratively repeated each time a new measurement is taken, and different clusters/prior patient exams may be identified once sufficient identifying/differentiating measurements have been taken and used in the clustering process. In other examples, the first measurements and any prior or subsequent measurements may be used in the processed described above in response to a user request for suggested diagnosis codes, findings, and/or further measurements, or the first measurements and any prior or subsequent measurements may be used in the process described above in response to an indication that the current exam is complete (e.g., the user indicates that all measurements have been taken).

At 408, method 400 includes displaying the identified diagnosis codes based on the query. Diagnosis codes and findings may be displayed in an interface on a display device, such as interface 200 of FIG. 2. In one example, the display device may be the same display device used to display the ultrasound images. In another example, a secondary display device may be used to display the interface of diagnosis codes and findings. For example, the process of reducing the database to the relevant measurements, doing dimensionality reduction, clustering and mapping the current exam into this space may identify the one or more clusters/prior patient exams as explained above, and send the diagnosis code(s) and/or finding(s) associated with each identified cluster/prior patient exam. In another example, the process of reducing the database to the relevant measurements, doing dimensionality reduction, clustering and mapping the current exam into this space may identify which diagnosis code(s) and/or finding(s) from the identified clusters/prior patient exams are most probable (most commonly occurring among the exams in the cluster(s)) based on the received measurements, and send those diagnosis code(s) and/or finding(s). In this way, the result of the lookup/search of the current measurements may be a list of the diagnosis codes from the cluster(s) which most closely match the measurements and measurement values of the current measurements, which may then be presented to the user.

At 410, method 400 includes calculating if an additional measurement would increase a diagnosis confidence. Based on the current measurement and related measurements found by the lookup, a calculation may be performed to determine if one or more additional measurements would increase a diagnosis confidence. If a plurality of additional measurements may be determined to increase a diagnosis confidence, the calculation may determine a minimum amount of additional measurements that may increase diagnosis confidence. In one example, the diagnosis confidence may be considered to be sufficiently increased if an amount of suggested diagnosis codes and findings is reduced by a predetermined amount or percentage. In another example, the diagnosis confidence may be considered to be sufficiently increased if an amount of increase in the diagnosis confidence is greater than a predetermined confidence threshold. The calculation of whether the additional measurement(s) would increase a diagnosis confidence may be performed by a separate computing device, e.g., the computing device storing the database and performing data reduction and clustering and/or in communication with the devices doing this, such as the suggestion system 124, at least in some examples.

At 412, method 400 includes suggesting taking additional measurements based on the calculations from 410. The suggested additional measurements may be selected based on a similar clustering process as described above, including the measurements of the current exam and iteratively include additional measurements. The additional measurements are correlated to the value of the first measurement, as the additional measurements are suggested based on the first measurement and value of the first measurement (e.g., to narrow down the possible clusters/prior patient exams suggested based on the first measurement and/or to differentiate between two or more identified clusters/prior patient exams identified based on the first measurement). The additional measurements may be suggested to the user before the user takes the additional measurements.

To identify an additional measurement that may narrow down the possible diagnosis codes, a second set of exams is extracted from the database, where each exam in the second set of exams has at least the same measurements as the current exam and an additional, not-yet-taken measurement. The second set of exams is clustered into at least two clusters, similar to the clustering process described above (which may include the application of the PCA to reduce the dimensionality of the data in the exams). Then, for each cluster of the at least two clusters of the second set of exams, a subset of diagnosis codes of the exams included in that cluster is extracted. For example, the top five most occurring diagnosis codes in that cluster may be extracted, for each cluster. A differentiating score is calculated based on the extracted subsets of diagnosis codes. The differentiating score may indicate how similar or different each cluster is to each other cluster. For example, a low differentiating score may indicate that the clusters are similar to each other, based on the clusters have the same diagnosis codes. A high differentiating score may indicate that the clusters are different from each other, based on the clusters having different diagnosis codes. If it is determined that the differentiating score is higher than a threshold, the additional measurement may be suggested to the user. The threshold may be a differentiating score calculated for the exams that include at least the measurements of the current exam (without including the additional measurement).

At 416, method 400 includes determining if additional measurements are taken. A user may optionally choose to make additional measurements based on user preference or possible suggestions from 412. The determination of whether additional measurements are taken may be based on received user input (e.g., user input placing measurement points). If additional measurements are not taken, method 400 may continue to 420, which includes displaying selectable diagnosis codes and findings.

If additional measurements are taken, at 418, method 400 includes querying the database to determine diagnosis codes to suggest to the user based on the measurements, which may include the first set of measurements and the new, additional measurements. The same criteria adhered to by the lookup may be adhered to when narrowing down the related or equivalent measurements. For example, the first measurements and the additional measurement(s) that are taken (including the type of measurements and values of the measurements) may be sent to through the process of reducing the database of prior exams to only exams with the relevant measurements, then doing dimensionality reduction, then non-supervised clustering, then converting the values of current exam to the dimensionality reduced space and identifying which cluster(s) of the database of clustered measurements (e.g., which prior patient exams) are most similar to the current exam, based on the first measurements and additional measurement(s). When one or more clusters/prior patient exams are identified, the most common diagnosis codes/findings in the identified clusters/prior patient exams may be extracted and presented to the user. As more and more measurements are taken, the list of probable diagnosis codes or findings may be narrowed down. For example, after the first set of measurements, a first list of tags (each indicating a diagnosis code or finding) may be returned to the user, where the first list of tags includes a subset of a plurality of possible tags and excludes remaining tags from the plurality of possible tags. After a second measurement is taken, a second list of tags may be returned to the user, where the second list of tags includes one or more tags from the first list of tags and excludes remaining tags from the first list of tags. In some examples, when the second measurement is received, only tags from the list of tags may be queried, which may reduce the processing power necessary to identify the second list of tags and lower the amount of time needed to identify the second list of tags.

Upon performing the new search with the additional measurements, method 400 continues back to 408 to display the identified diagnosis codes and/or findings as suggested by the process involving clustering of prior exam measurements. This process of suggesting measurements and diagnosis codes may be iteratively repeated as more measurements are taken. For example, after the additional measurements are suggested, a second user input may be received from the user and a second measurement of the ultrasound image may be determined based on the second user input. The second measurement and a value of the second measurement may be sent to the database of clustered measurements. A third suggested measurement may be received from the database, where the third suggested measurement is correlated to the value of the first measurement and the value of the second measurement. The third suggested measurement may be suggested to the user before the user performs the third measurement. Further, it will be appreciated that the measurements that are taken and suggested, as well as the suggested diagnosis codes and findings, may apply to the currently displayed ultrasound image and/or other ultrasound images in the current patient exam. For example, echocardiograms may typically include a plurality of images taken at different anatomical views (e.g., PLAX, PSAX, A4C, etc.), with each view imaged in one or more imaging modes (e.g., B mode, M mode, Doppler, etc.). Thus, different measurements may be performed on different images, and as such, the ultrasound image that is currently displayed may change as method 400 progresses. Further, when method 400 is executed during an active imaging session, in addition or alternative to suggesting additional measurements, additional views and/or imaging modes may also be suggested to ensure a complete exam is performed, which may be guided by the measurements already taken.

Returning to 416, if additional measurements are not taken and/or if the user indicates that all measurements are complete, method 400 proceeds to 420, where method 400 includes displaying selectable diagnosis codes and findings. The displayed list of diagnosis codes and findings may be all possible diagnosis codes and findings for the type of exam being performed. In other examples, the displayed diagnosis codes and findings may be narrowed down based on the measurements that have been taken and suggested diagnosis codes/findings determined based on the measurements. From the displayed list of selectable diagnosis codes and findings, the user may select one or more diagnosis codes and/or findings to be saved as part of the patient exam, which may be influenced by the suggested diagnosis codes and/or findings.

At 422, method 400 includes applying selected diagnosis codes and/or findings to current patient exam data (e.g., to the current patient exam). The user may select any diagnosis codes and/or findings to apply the associated data with the current patient exam data. The user may also choose to apply user defined tags based on the current patient exam or user preference.

Thus, method 400 provides for identification of possible diagnosis codes and further measurements, based on a set of measurements for a current exam. The possible diagnosis codes and/or measurements may be identified from prior exams that have been clustered based on the measurements and measurement values in the prior exams. However, the clustering can only happen once at least some measurements have been performed in the current exam, as the measurements of the current exam are used to narrow down the exams to be included in the clustering. Thus, the clustering occurs during the current exam—either triggered by the user, or automatically (when a measurement is finished). Additionally, clustering can only happen on low dimensionality, such as <8 dimensions (due to the high dimensionality). Thus, the dimensionality may be before clustering can happen. The dimensionality may be reduced using Principal Component Analysis (PCA). The PCA may run on all exams in database containing the same measurements as currently in the local exam.

The clustering itself may be by k-means/k-nearest neighbors or by multiple correspondence analysis (MCA) clustering. MCA has the benefit of allowing some exams to not be forced to be part of a cluster, which may reduce noise. After the clustering has happened, the cluster that the current exam measurements (most closely) belongs to may be identified. To achieve this, the PCA values for the current exam measurement values may be calculate based on the same PCA transformation as arrived upon for the exams of the database. Once a cluster is identified, a subset of the diagnosis codes from the exams of cluster (e.g., the top 5 most occurring diagnosis codes/findings of the exams in the cluster) may be suggested to the user.

After this, in order to suggest the next measurement, each candidate measurement may be evaluated as follows:

f_1: Get the N measurement IDs in current exam so far

f_2: For each candidate measurement m_i:

f_3: Do PCA analysis on all exams in database which have these N measurements+m_i

f_4: Do k means clustering with a target of W clusters

f_5: Calculate the score for “discerning between diagnosis tags”

f_6: Once the score is calculated for all candidate measurements—suggest the candidate measurement m_i with the highest score. Alternative to f_6, after f_5, if the score for N+m_i is better than the score for N, m_i may be suggested as the next measurement.

FIG. 5 shows an example database structure 500, where one or more diagnosis tags may be identified based on a plurality of measurements. The database structure 500 may be generated during a current patient exam. Structure 500 may be one example illustrating how the database (or system executing the database) determines which diagnosis codes to suggest to the user, described above with respect to FIG. 4, but other approaches for making suggestions based on the database may be implemented without departing from the scope of the disclosure.

In structure 500, an exam column 502 may include all exams stored in the database. A plurality of measurement columns, such as measurement column 504, may include each possible measurement that is present in at least one exam in the database. In FIG. 5, five measurement columns are shown, one for each of five different measurements (e.g., measurements A-E). Structure 500 also includes a plurality of diagnosis tag columns, such as diagnosis tag column 506, including one diagnosis tag column for each diagnosis tag that is present in at least one exam in the database. In FIG. 5, four diagnosis tag columns are shown, one column for each of four different tags (e.g., tags 1-4).

The measurements (and measurement values) as well as diagnosis tags from each exam may be populated in the structure 500. For example, for a first exam (exam 1), a measurement was taken for each of measurements A-D, and diagnosis tags 1-3 were associated with the first exam. Values/indicators for each of the measurements and diagnosis tags are populated in the row for the first exam.

When the structure 500 is queried, the measurements from the current exam are used to identify similar exams in the database, based on which exams have the same measurements. For example, N measurements in the current exam so far may be N=3, with the measurements being a, b and c. All exams in the database which have these N measurements are identified. In the example of FIG. 5, the identified exams may be exams 1, 2, 10, and 11 (4 exams in total).

Dimensionality reduction by PCA analysis may then be performed on all exams in database which have these N measurements (e.g., the four exams identified above). For example, the PCA analysis may return PCA_1 and PCA_2 as the two first principal components/dimensions.

Clustering is then performed, with the exams clustered/plotted along the dimensions identified by the PCA analysis. The clustering may be k means clustering with a target of W clusters, which in the example of FIG. 5 may be a target of two clusters. An example of the clustering after PCA analysis is shown in FIG. 6, which includes a plot 600 with the PCA_1 plotted along the vertical axis and the PCA_2 plotted along the horizontal axis. The clustering may result in two clusters, with two exams in each cluster (e.g., the exams in a first cluster shown in white and the exams in a second cluster shown in black).

PCA values are calculated for the N measurements of the current exam and plotted with the clustered exams. In this example, the current exam point in the PCA space may be positioned closest to the black dots (e.g., the second cluster). Thus, the current exam belongs to the second (black) cluster. The top portion (e.g., five) most occurring diagnosis codes/tags of the exams in the cluster are then identified. In this example, exam 1 and exam 2 are in the second cluster. Thus, the most commonly occurring tags (from exams 1 and 2 as shown in FIG. 5) are tag 1, tag 2, and tag 3 (e.g., “x” “o” and “.”).

Additional measurements may also be suggested. To identify additional measurements, a score for “discerning between diagnosis tags” may be calculated for the N measurements. For each additional candidate measurement m_i (e.g., a measurement from the database that has not been taken yet, such as Meas D), a PCA analysis is performed on all exams in database which have the N measurements+m_i. For example, in the example shown in FIG. 5, exam 1 and exam 2 may be selected. Clustering (e.g., k means clustering) is then performed with a target of W clusters. The score for “discerning between diagnosis tags” is calculated (see below). If the score is better (e.g., higher) than for a score calculated with only the N measurements: suggest this measurement (e.g. Meas D) as the next measurement. If the score is not better (e.g., not higher) than for score for the N measurements, then the method advances to assess the next candidate measurement m_i.

To calculate the score for “discerning between diagnosis tags,” for each cluster, the top 5 occurring diagnosis tags for the exams in this cluster are identified/extracted. The score is calculated based on how unique the 5 tags from one cluster are compared to all other clusters. W is number of clusters. The score is assigned based on how often the tag occurs in the exams of the cluster. For each totally unique tag, e.g., mentioned only one cluster, W points is assigned. For each tag shared between two clusters only: W−1 points is assigned. For each tag shared by three clusters only: W−2 points is assigned. The final score is calculated by dividing the points by W*5. For example, the highest score possible (e.g., if all W clusters have 5 unique tags which no other clusters have) is (W*5)/(W*5)=1.0 The lowest score possible (e.g., if all clusters have the same 5 tags) is (W*1)/(W*5)=⅕.

By calculating the score based on the current measurements, then recalculating the score including one additional measurement in an iterative fashion, a measurement that increases the score may be quickly identified and suggested.

By structuring the results of the clustering as shown by the structure 500 and/or returning database queries when a set of measurements is received according to the structure 500, several advantages may be achieved. The structure 500 may demand minimized memory usage, as only pertinent data from the exams and clustering may be saved (e.g., other information from the exams may be discarded) and the data may be structured in an efficient manner. When a query is performed to identify tags and/or measurements from a received set of measurements, the results may be returned quickly, particularly as the list of possible tags narrows with each additional measurement. Further, processing power may be reduced by providing for easy identification of relevant measurements and tags, which may also be used to assist in identifying additional, diagnostically relevant measurements to be performed.

In one example, the current patient exam may include the same measurements as previous patient exams including a given diagnosis code (e.g., tag X), so measurement values relating to the current patient exam may be compared to measurement values from previous exams including the given diagnosis code, bounded by the clustering algorithm as described with respect to FIG. 4, in order to determine if the current patient exam should also include the given diagnosis code. When comparing measurement values, if a measurement value from the current patient exam is not in the range of values bounded by the cluster associated with the measurement, the given diagnosis code may not be suggested. In this example, each measurement associated with a tag (as determined by the database) may be present in the current exam, with values in predefined ranges, in order for that tag to be suggested. However, in other examples, if a measurement is missing or has a value outside a predefined range, the tag may still be suggested, but with a lower confidence.

In another example, the current patient exam may not include a measurement that is included in previous patient exams that include a plurality of diagnosis codes currently associated with the current patient exam, so probabilities of each diagnosis code being associated with the current patient exam after including the measurement may be compared for all diagnosis codes currently associated with the current patient exam that include the measurement currently not included in the current patient exam. If any probabilities of any diagnosis codes being associated with the current patient exam after including the measurement vary by an amount exceeding a variance threshold, it may be determined that the measurement may be suggested to the user to narrow down a list of possible diagnosis codes to include in the current patient exam.

While the suggestion system, database of clustered measurements, and methods for clustering the data and suggesting possible diagnoses and measurements to a user have been described herein with respect to an ultrasound system, it is to be appreciated that the systems and methods described herein may be applied to other types of medical images, including but not limited to magnetic resonance images, computed tomography images, X-ray images, visible light images, and the like.

A technical effect of suggesting possible diagnoses and/or measurements for a patient exam, based on one or more measurements of anatomical features in medical images of the patient, using a database of exams with matching exams clustered according to a current set of measurements is that accurate diagnoses may be provided and/or a level of confidence in a diagnosis may be increased, improving patient care. A further technical effect is that the database may be implemented on a variety of devices and allow for suggested diagnoses and/or measurements in a variety of clinical settings, while reducing the processing power and memory demanded of other computer-aided diagnosis systems.

The disclosure also provides support for a method for a user interface of a medical imaging system, comprising: receiving first user input from a user and determining a first set of measurements on a medical image based on the first user input, sending the first set of measurements to a database of exams, receiving a subset of exams from the database, the subset based on the first set of measurements, determining, after clustering the subset of exams, a second measurement, and suggesting the second measurement to the user before the user performs the second measurement. In a first example of the method, the method further comprises: receiving second user input from the user and determining the second measurement of the medical image based on the second user input, sending the second measurement to the database of exams, receiving a second subset of exams from the database, the second subset based on the first set of measurements and the second measurement, determining, after clustering the second subset of exams, a third measurement, and suggesting the third measurement to the user before the user performs the third measurement. In a second example of the method, optionally including the first example, the method further comprises: receiving second user input from the user and determining the second measurement of the medical image based on the second user input, sending the second measurement and a value of the second measurement to the database of exams, receiving a second subset of exams from the database, the second subset based on the first set of measurements and a value of each measurement of the first set of measurements and the second measurement and the value of the second measurement, determining, after clustering the second subset of exams, a suggested first tag indicating a possible diagnosis code or finding based on each value of the first set of measurements and the value of the second measurement, and displaying the suggested first tag. In a third example of the method, optionally including one or both of the first and second examples, upon clustering the subset of exams, the first tag and a second tag are identified, and wherein the second measurement is suggested to differentiate the first tag and the second tag. In a fourth example of the method, optionally including one or more or each of the first through third examples, the first tag and the second tag are identified by mapping the first set of measurements and the value of each measurement of the first set of measurements to a cluster of exams, and then identifying that the first tag and the second tag are the most occurring tags for the exams in this cluster. In a fifth example of the method, optionally including one or more or each of the first through fourth examples, the method further comprises: determining that the first set of measurements includes more than six measurements, and in response, performing a dimensionality reduction on the subset of exams based on the first set of measurements, using Principal Component Analysis, prior to the clustering of the subset of exams. In a sixth example of the method, optionally including one or more or each of the first through fifth examples, the method further comprises: performing the dimensionality reduction on the first set of measurements and the value of each measurement of the first set of measurements prior to the mapping. In a seventh example of the method, optionally including one or more or each of the first through sixth examples, the medical image is of a patient, and further comprising, responsive to receiving a third user input selecting the first tag, saving the first tag as part of a report for the patient, the report further including the medical image, the first set of measurements, the value of each measurement of the first set of measurements, the second measurement, and the value of the second measurement. In an eighth example of the method, optionally including one or more or each of the first through seventh examples, the medical image is of a patient, and further comprising displaying one or more additional tags and responsive to a third user input selecting a tag of the one or more additional tags, saving the selected tag as part of a report for the patient, the report further including the medical image, the first set of measurements, the value of each measurement of the first set of measurements, the second measurement, and the value of the second measurement. In a ninth example of the method, optionally including one or more or each of the first through eighth examples, upon receiving the first set of measurements and the value of each measurement of the first set of measurements, a first subset of possible tags is identified from among a plurality of possible tags listed in the database and all other tags in the plurality of possible tags are excluded, upon receiving the second measurement and the value of the second measurement, a second subset of possible tags is identified from among the first subset of possible tags and all other tags in the first subset of tags are excluded, and further comprising receiving the second subset of tags and displaying the second subset of tags. In a tenth example of the method, optionally including one or more or each of the first through ninth examples, the subset of exams includes only exams from the database that include at least the first set of measurements.

The disclosure also provides support for a method, comprising: receiving a first set of measurements of an anatomical feature in a medical image and a value of each measurement of the first set of measurements, mapping the first set of measurements and the value of each measurement of the first set of measurements to at least a first tag and a second tag via a set of clustered exams, each tag indicative of a diagnosis code or finding relating to the anatomical feature in the medical image, determining that a second measurement of the anatomical feature will differentiate between the first tag and the second tag, and in response, outputting a suggestion that the second measurement should be performed. In a first example of the method, the set of clustered exams is extracted from a database that comprises data from a plurality of prior patient exams, the set of clustered exams clustered based on the first set of measurements and the value of each measurement of the first set of measurements. In a second example of the method, optionally including the first example, the data in the database includes data from only prior patient exams that include less than a first threshold number of tags, a number of measurements within a second threshold range, and an indication that the prior patient exam is complete. In a third example of the method, optionally including one or both of the first and second examples, the prior patient exams include user-defined tags and non-user-defined tags, and wherein only consistently-used user-defined tags are included in the database, wherein consistently-used user-defined tags are identified based on a frequency of usage and/or time period of usage. In a fourth example of the method, optionally including one or more or each of the first through third examples, mapping the first set of measurements and the value of each measurement of the first set of measurements to at least the first tag and the second tag comprises: identifying the set of exams based on each exam in the set of exams having the first set of measurements, clustering the set of exams into at least two clusters, mapping the first set of measurements and the value of each measurement of the first set of measurements to a cluster of the at least two clusters, and determining that the first tag and the second tag are included in exams of the cluster more frequently than any other tags. In a fifth example of the method, optionally including one or more or each of the first through fourth examples, determining that the second measurement will differentiate between the first tag and the second tag comprises: identifying a second set of exams based on each exam in the second set of exams having the first set of measurements and the second measurement, clustering the second set of exams into at least two clusters, for each cluster of the at least two clusters of the second set of exams, extracting a subset of diagnosis tags of the exams included in that cluster, calculating a differentiating score based on the extracted subsets of diagnosis tags, determining that the differentiating score is greater than a threshold, and in response, determining that the second measurement will differentiate between the first tag and the second tag.

The disclosure also provides support for a system, comprising: a memory storing instructions, and a processor configured to execute the instructions to: receive a set of measurements of patient anatomical features present in one or more medical images of a patient, each measurement having a respective value, identify a list of tags correlated with the set of measurements, each tag in the list of tags indicating a respective diagnosis code or finding, the list of tags identified from among a plurality of possible tags from a set of exams clustered based on the set of measurements, and output the list of tags for display on a display device. In a first example of the system, the instructions are further executable to identify an additional measurement to be performed based on the set of measurements and the list of tags and output a suggestion that the additional measurement be performed for display on the display device. In a second example of the system, optionally including the first example, the instructions are further executable to receive the additional measurement and a value of the additional measurement, as measured by a user on the one or more medical images, identify a narrowed list of tags based on the list of tags and value of the additional measurement, and output the narrowest list of tags for display on the display device.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for a user interface of a medical imaging system, comprising:
receiving first user input from a user and determining a first set of measurements on a medical image based on the first user input;
sending the first set of measurements to a database of exams;
receiving a subset of exams from the database, the subset based on the first set of measurements;
determining, after clustering the subset of exams, a second measurement; and
suggesting the second measurement to the user before the user performs the second measurement.

2. The method of claim 1, further comprising:
receiving second user input from the user and determining the second measurement of the medical image based on the second user input;
sending the second measurement to the database of exams;
receiving a second subset of exams from the database, the second subset based on the first set of measurements and the second measurement;
determining, after clustering the second subset of exams, a third measurement; and
suggesting the third measurement to the user before the user performs the third measurement.

3. The method of claim 1, further comprising:
receiving second user input from the user and determining the second measurement of the medical image based on the second user input;
sending the second measurement and a value of the second measurement to the database of exams;
receiving a second subset of exams from the database, the second subset based on the first set of measurements and a value of each measurement of the first set of measurements and the second measurement and the value of the second measurement;
determining, after clustering the second subset of exams, a suggested first tag indicating a possible diagnosis code or finding based on each value of the first set of measurements and the value of the second measurement; and
displaying the suggested first tag.

4. The method of claim 3, wherein upon clustering the subset of exams, the first tag and a second tag are identified, and wherein the second measurement is suggested to differentiate the first tag and the second tag.

5. The method of claim 4, wherein the first tag and the second tag are identified by mapping the first set of measurements and the value of each measurement of the first set of measurements to a cluster of exams, and then identifying that the first tag and the second tag are the most occurring tags for the exams in this cluster.

6. The method of claim 5, further comprising determining that the first set of measurements includes more than six measurements, and in response, performing a dimensionality reduction on the subset of exams based on the first set of measurements, using Principal Component Analysis, prior to the clustering of the subset of exams.

7. The method of claim 6, further comprising performing the dimensionality reduction on the first set of measurements and the value of each measurement of the first set of measurements prior to the mapping.

8. The method of claim 3, wherein the medical image is of a patient, and further comprising, responsive to receiving a third user input selecting the first tag, saving the first tag as part of a report for the patient, the report further including the medical image, the first set of measurements, the value of each measurement of the first set of measurements, the second measurement, and the value of the second measurement.

9. The method of claim 3, wherein the medical image is of a patient, and further comprising displaying one or more additional tags and responsive to a third user input selecting a tag of the one or more additional tags, saving the selected tag as part of a report for the patient, the report further including the medical image, the first set of measurements, the value of each measurement of the first set of measurements, the second measurement, and the value of the second measurement.

10. The method of claim 3, wherein upon receiving the first set of measurements and the value of each measurement of the first set of measurements, a first subset of possible tags is identified from among a plurality of possible tags listed in the database and all other tags in the plurality of possible tags are excluded;
upon receiving the second measurement and the value of the second measurement, a second subset of possible tags is identified from among the first subset of possible tags and all other tags in the first subset of tags are excluded; and
further comprising receiving the second subset of tags and displaying the second subset of tags.

11. The method of claim 1, where the subset of exams includes only exams from the database that include at least the first set of measurements.

12. A method, comprising:
receiving a first set of measurements of an anatomical feature in a medical image and a value of each measurement of the first set of measurements;
mapping the first set of measurements and the value of each measurement of the first set of measurements to at least a first tag and a second tag via a set of clustered exams, each tag indicative of a diagnosis code or finding relating to the anatomical feature in the medical image;
determining that a second measurement of the anatomical feature will differentiate between the first tag and the second tag, and in response, outputting a suggestion that the second measurement should be performed.

13. The method of claim 12, wherein the set of clustered exams is extracted from a database that comprises data from a plurality of prior patient exams, the set of clustered exams clustered based on the first set of measurements and the value of each measurement of the first set of measurements.

14. The method of claim 13, wherein the data in the database includes data from only prior patient exams that include less than a first threshold number of tags, a number of measurements within a second threshold range, and an indication that the prior patient exam is complete.

15. The method of claim 14, wherein the prior patient exams include user-defined tags and non-user-defined tags, and wherein only consistently-used user-defined tags are included in the database, wherein consistently-used user-defined tags are identified based on a frequency of usage and/or time period of usage.

16. The method of claim 13, wherein mapping the first set of measurements and the value of each measurement of the first set of measurements to at least the first tag and the second tag comprises:
identifying the set of exams based on each exam in the set of exams having the first set of measurements;
clustering the set of exams into at least two clusters;
mapping the first set of measurements and the value of each measurement of the first set of measurements to a cluster of the at least two clusters; and
determining that the first tag and the second tag are included in exams of the cluster more frequently than any other tags.

17. The method of claim 16, wherein determining that the second measurement will differentiate between the first tag and the second tag comprises:
identifying a second set of exams based on each exam in the second set of exams having the first set of measurements and the second measurement;
clustering the second set of exams into at least two clusters;
for each cluster of the at least two clusters of the second set of exams, extracting a subset of diagnosis tags of the exams included in that cluster;
calculating a differentiating score based on the extracted subsets of diagnosis tags;
determining that the differentiating score is greater than a threshold, and in response, determining that the second measurement will differentiate between the first tag and the second tag.

18. A system, comprising:
a memory storing instructions; and
a processor configured to execute the instructions to:
receive a set of measurements of patient anatomical features present in one or more medical images of a patient, each measurement having a respective value;
identify a list of tags correlated with the set of measurements, each tag in the list of tags indicating a respective diagnosis code or finding, the list of tags identified from among a plurality of possible tags from a set of exams clustered based on the set of measurements; and
output the list of tags for display on a display device.

19. The system of claim 18, wherein the instructions are further executable to identify an additional measurement to be performed based on the set of measurements and the list of tags and output a suggestion that the additional measurement be performed for display on the display device.

20. The system of claim 19, wherein the instructions are further executable to receive the additional measurement and a value of the additional measurement, as measured by a user on the one or more medical images, identify a narrowed list of tags based on the list of tags and value of the additional measurement, and output the narrowest list of tags for display on the display device.
